(19) United States
(12) Patent Application Publication
(10) Pub. No.: US 2023/0009821 A1
(43) Pub. Date: Jan. 12, 2023

(54) EVALUATION AND CONTROL SYSTEM FOR CORNEA AND INTRAOCULAR REFRACTIVE SURGERY

(71) Applicants: Arturos S. Chayet, La Jolla, CA (US); Erik Navas, Tijuana (MX)

(72) Inventors: Arturos S. Chayet, La Jolla, CA (US); Erik Navas, Tijuana (MX)

(21) Appl. No.: 17/856,459

(22) Filed: Jul. 1, 2022

Related U.S. Application Data
(60) Provisional application No. 63/311,784, filed on Feb. 18, 2022.
(60) Provisional application No. 63/218,179, filed on Jul. 2, 2021.

(51) Int. Cl. A61B 3/103; A61B 3/00

(52) U.S. Cl. CPC A61B 3/1035; A61B 3/0025

(57) ABSTRACT

Techniques for lens design and evaluation involve configuring a rule comprising one of a “with the rule” and “against the rule”, configuring a cylinder comprising one of a “positive cylinder” and a “negative cylinder”, and utilizing the rule and the cylinder in one or both of a residual astigmatism metric algorithm and spherical equivalent metric algorithm to generate a discrete metric values each corresponding to ranges of residual refractive error.

### CROSS REFERENCE TO RELATED APPLICATIONS

This application claims priority and benefit under 35 USC 119 to U.S. Application No. 63/218,179, filed on Jul. 2, 2021, and to U.S. Application No. 63/311,784, filed on Feb. 18, 2022, the contents each of which are incorporated herein by reference in their entirety.

### BACKGROUND

Astigmatism in vision results from refractive errors caused by focusing problems. By some estimates approximately 33 percent of the U.S. population has some degree of astigmatism and that 70 percent of vision prescriptions written in the U.S. include astigmatism correction.

Prior methods for evaluation and control in cornea and intraocular refractive surgery procedures include the Alpins method, which uses vector mathematics to determine a goal for astigmatism correction and analyze factors involved if treatment fails to reach that goal. The Alpins method is complicated and not easy or often practical to use by physicians in the field or understood easy to understand by patients. There is therefore a need for more user friendly and more computationally efficient methods in this field.

US Patent U.S. Pat. No. 6,086,579A discloses determining a preoperative astigmatism, defining an aimed astigmatism and determining an achieved astigmatism following initial surgery. The astigmatism values are initially determined in a zero to 180 degree range and are doubled to convert them to a 360 degree range. An aimed induced astigmatism vector and a surgically induced astigmatism vector are calculated by vectorially adding the preoperative astigmatism respectively to the aimed astigmatism and the post-operative astigmatism. Magnitudes and angles of the vectors are related to one another and to their component values for providing fundamental information regarding the past surgery, improved performance of possible future surgery and also what alteration to the first surgical plan would have been required to have achieved the initial aimed astigmatism.

US Patent Publication No. US20120081661A1 describes a lens design algorithm wherein when a positive relative convergence, a negative relative convergence, a positive relative accommodation, a negative relative accommodation and a vertical fusional vergence, which are individual measurement values relating to binocular vision, are defined as relative measurement values, at least one of or both of the positive relative convergence and the negative relative convergence is included in an individual relative measurement value, and the optical design values for lenses are determined by optimizing binocular vision while using, as an evaluation function for the optimizing, a function obtained by adding binocular visual acuity functions including the relative measurement values as factors at respective evaluation points of an object.

Japanese application JPWO2002088828A1 describes a lens design method that takes into account eye movements (listing rules), and a merit function used in lens design optimization calculation processing includes a visual acuity evaluation function (log MAR) derived from a visual acuity measurement value, wherein the visual acuity evaluation function by a complex equation.

Japanese application JPWO2004018988A1 describes a lens design algorithm utilizing a correlation between visual acuity when viewed through an optical system and lateral chromatic aberration of the optical system, wherein when the visual acuity is expressed in logarithmic visual acuity, the log visual acuity is the magnification. The performance of the optical system is evaluated based on a correlation that becomes a proportional relationship that deteriorates substantially in proportion to chromatic aberration, or a correlation between the visual acuity substantially equivalent to this correlation and an optical value related to the lateral chromatic aberration.

US Patent No. U.S. Pat. No. 7,841,720B2 describes characterizing at least one corneal surface as a mathematical model, calculating the resulting aberrations of said corneal surfaces by employing said mathematical model, and selecting the optical power of the intraocular lens. From this information, an ophthalmic lens is modeled so a wavefront arriving from an optical system comprising said lens and corneal model obtains reduced aberrations in the eye.

US Patent Publication No. US20200383775A1 describes a method of designing an intraocular lens by providing a series of intraocular lenses of different net asphericity value, positioning a patient in front of a visual simulator of adaptive optics, emulating different intraocular lens profiles with different net asphericity value, realizing different simulations with different intraocular lens profiles through a visual test at different distances, selecting an optimal result of the visual test, and thereby determining the net asphericity value of the intraocular lens.

US Patent Publication No. US20100271591A1 describes a method of designing intraocular lenses utilizing a pseudoaphakic eye model, the definition of a merit function in multiple dimensions, which analytically connects the quality of the image on the retina to the optical and geometric parameters of the pseudoaphakic eye model, and the algorithm optimisation of the previous merit function using analytical and numerical methods in order to obtain one or more minimum globals which provide the optimal parameters of the intraocular lens for the pseudoaphakic eye model.

Russian Patent No. RU2629532C1 describes the clinical assessment of the lens state by determination of a set of diagnostic criteria including lens transparency, refraction, accommodation, lens topography and capsular-ligament support state. The state of each criterion is assessed in points, and the obtained points are summarized. According to the number of obtained points, the anatomical and functional state of the lens is determined as high, corresponding to normal, average, with a partial loss of functions, which shows dynamic observation and symptomatic treatment, or low, with a significant loss of functions, which shows the replacement of the lens with the intraocular lens.

Japanese Patent Application No. JP2007000255A describes a selection system of a best trial lens in the orthokeratology specifications based on a fitting evaluation. The selection system executes a counselling, objective examinations such as an curvature radius measurement/a refraction measurement/an intraocular pressure measurement by an autorefractometer, subjective examinations such as a visual acuity measurement of naked eye/fully corrected visual acuity measurement, anterior ocular segment examination/examinations of the fundus oculi and lacrimal fluid accompanied with the eye section, and basic examinations such as a measurement of cornea shape before the installation of a lens by a corneal topographer for obtaining data items classified into categories.

US Patent No. U.S. Pat. No. 8,746,882B2 describes selecting an optimal intraocular lens (IOL) from a plurality of IOLs for implanting in a subject eye, including measuring anterior corneal topography (ACT), axial length (AXL), and anterior chamber depth (ACD) of a subject eye; selecting a default equivalent refractive index depending on preoperative patient's stage or calculating a personalized value or introducing a complete topographic representation if posterior corneal data are available; creating a customized model of the subject eye with each of a plurality of identified intraocular lenses (IOL) implanted, performing a ray tracing through that model eye; calculating from the ray tracing a RpMTF or RMTF value; and selecting the IOL corresponding to the highest RpMTF or RMTF value for implanting in the subject eye.

Australian Patent No. AU2012224545B2 describes determination of the post-operative position of an intraocular lens in an eye of a patient undergoing lens replacement surgery, which involves determining the position of the existing crystalline lens in the pre-operative eye of the patient and using that information and a single numerical constant to predict the post-operative intraocular lens position. Japanese Patent No. JP5335922B2 describes methods for designing and implanting a customized intra-ocular lens (IOL) utilizing an eye analysis module that analyzes a patient's eye and generates biometric information relating to the eye. The system also includes eye modeling and optimization modules to generate an optimized IOL model based upon the biometric information and other inputted parameters representative of patient preferences. The system further includes a manufacturing module configured manufacture the customized IOL based on the optimized IOL model. In addition, the system can include an intra-operative real time analyzer configured to measure and display topography and aberrometry information related to a patient's eye for assisting in proper implantation of the IOL.

US Application No. US20160346047A1 a method for guiding an astigmatism correction procedure on an eye of a patient. A photosensor records a pre-operative still image of an ocular target surgical site of the patient. A a real-time multidimensional visualization of the ocular target surgical site is produced during an astigmatism correction procedure. A virtual indicium is determined that includes data for guiding the astigmatism correction procedure. The pre-operative still image is utilized to align the virtual indicium with the multidimensional visualization such that the virtual indicium is rotationally accurate.

European Patent No. EP3522771B1 describes a process for designing and evaluating intraocular lenses, by generating a first plurality of eye models, wherein each eye model corresponds to a patient using data that includes constant and customized values, including customized values of a first intraocular lens; simulating first outcomes provided by the first intraocular lens in the first plurality of eye models; creating a database of the first outcomes; generating a second plurality of eye models, wherein the first intraocular lens in the first plurality of eye models is substituted with a second intraocular lens; simulating second outcomes provided by the second intraocular lens in the second plurality of eye models; and comparing the first outcomes with the second outcomes, evaluating the first or second intraocular lens on the basis of the compared outcomes.

U.S. patent Ser. No. 10/734,114B2 describes a customer diagnostic center configured to generate customer examination data pertaining to an examination of a customer's eye. The customer diagnostic center provides a user interface for communicating with a customer and ophthalmic equipment for administering tests to the customer. A diagnostic center server is configured to receive the customer examination data from the customer diagnostic center over a network and allow the customer examination data to be accessed by an eye-care practitioner. A practitioner device associated with the eye-care practitioner is configured to receive the customer examination data from the diagnostic center server and display at least a portion of the customer examination data to the eye-care practitioner. Customer evaluation data is generated pertaining to the eye-care practitioner's evaluation of the customer examination data. An eye health report is provided to the customer via the network.

US Patent No. U.S. Pat. No. 9,931,199B2 describes a surgical method on the eye of a patient that includes measuring a surface of a cornea of the eye to acquire eye topography data. The method includes, based on the eye topography data, selecting a topographic pattern from topographic patterns displayed in a graphical user interface. The method includes entering vision corrective parameters for the eye of the patient into the graphical user interface. The method includes actuating a processing module to obtain a surgical plan based on the selected topographic pattern and the entered vision corrective parameters.

US Patent Application 20190290423A1 describes method for selecting toric intraocular lenses (IOL) and relaxing incision for correcting refractive error. The one or more toric IOL and relaxing incision combinations can be used for off-axis correction of refractive errors such as astigmatism. The disclosure provides a method for selecting toric IOL and relaxing incision combinations that have combined astigmatism correcting powers and off-axis positions or orientations of the astigmatism correcting axes of the toric IOL and relaxing incision that are effective to yield lower residual astigmatism than on axis correction methods. The toric IOL and relaxing incision combinations also allow the user to avoid incisions that will radially overlap with a cataract incision thereby provided improved outcomes.

Chinese Patent No. CN1192132A describes a method of surgically treating an eye of a patient to correct astigmatism in which values of astigmatism are measured topographically and refractively, and limit values of targeted induced astigmatism for the topographically and refractively measured astigmatism values are obtained by summating the topographical value of astigmatism with the refractive value of astigmatism and vice versa. Respective target values of astigmatism for refraction and topography based on the limit values are obtained and surgical treatment is effected with a target induced astigmatism which is intermediate the limit values and provided respective topographical and refractive non-zero target astigmatism values whose sum is a minimum.

Canadian Patent No. CA2968687A1 describes techniques in which a topographic parameter is determined in each hemidivision of the eye by considering the topography of reflected images from a multiplicity of illuminated concentric rings of the cornea. A simulated spherocylinder is produced to fit into each ring and conform to the topography thereof from which a topographic parameter for each ring can be obtained. All of the topographic parameters of each ring are combined and a mean summated value is obtained representing magnitude and meridian of each hemidivision. From these parameters, a single topographic value for the entire eye (CorT) can be found as well as a value representing topographic disparity (TD) between the two hemidivisions. The topography values for the hemidivisions are used in a vector planning system to obtain treatment parameters in a single step operation.

US Patent No. U.S. Pat. No. 8,678,587B2 describes techniques in which a topographic parameter is determined in each semi-meridian of the eye by considering the topography in each of three concentric zones from the central axis at 3 mm, 5 mm, and 7 mm and assigning weighting factors for each zone, By selectively treating the weighted values in the three zones, parameters of magnitude and meridian can be obtained for each semi-meridian. From these parameters, a single topographic value for the entire eye (CorT) can be found as well as a value representing topographic disparity (TD) between the two semi-meridians. The topography values for the semi-meridians are used in a vector planning system to obtain treatment parameters in a single step operation.

As these prior approaches demonstrate, it has proven to be exceedingly challenging for many years to develop computationally and procedurally efficient methods in this field that also provide suitable clinical accuracy.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A—FIG. 1D depict normal vision and astigmatism.

FIG. 2A—FIG. 2C depict characterization of astigmatism with and against the rule.

FIG. 3 depicts a vision analysis system 300 in one embodiment.

FIG. 4 depicts an algorithmic mapping of uncorrected distance visual acuity to a metric control, in accordance with one embodiment.

FIG. 5 depicts a client server network configuration 500 in accordance with one embodiment.

FIG. 6 depicts a cloud computing system 600 in accordance with one embodiment.

FIG. 7 depicts a machine 700 in the form of a computer system within which a set of instructions may be executed for causing the machine to perform any one or more of the methodologies discussed herein, according to an example embodiment.

### DETAILED DESCRIPTION

Disclosed herein are systems utilizing metric controls that relate visual acuity with manifest astigmatism and spherical equivalent, with the objective of rating refractive results for intraocular lens or corneal refractive surgery. The metric controls may be applied as a highly quantized setting (e.g., less than 10 and preferably 5 levels) for corrective lens selection or formation, based on residual refractive errors post refractive surgery, both corneal and intraocular (e.g. Phacoemulsification, LASIK, PRK, ICL)

The metric controls are generated from measurements of uncorrected visual acuity (distance, intermediate, or near) and manifest refraction. For each tier of the metrics a range of residual refractive astigmatism or spherical equivalent is given. The specific range is determinate by analyzing the amount of residual astigmatism or spherical equivalent that is necessary for visual acuity to change and that best correlates the metric value with visual acuity.

Astigmatism may be classified two ways: (1) against the rule and oblique, and (2) with the rule. With the rule is along the axis of the positive cylinder in a lens oriented at 90 degrees) (+−30°. Every other axis as oblique or against the rule. The algorithms map the two types of astigmatism into ranges, each assigned to a level (i.e., tier) and each associated with changes in visual acuity. Higher visual acuity correlates to a higher score or level in the system.

Spherical equivalent is calculated by the sum of the sphere power with half of the cylinder power. The algorithms define an amount and range of residual spherical equivalent for each of the tiers.

The disclosed mechanisms exhibit a reduction in procedural and computational complexity over prior approaches and enable the accumulation of metrics of success for lens selection over a wide range of patient characteristics. These accumulated metrics in turn enable greater precision of the lens design, selection, and evaluation algorithms, leading to a positive feedback cycle of lens design, manufacturing, and deployment. For example the disclosed mechanisms obviate the need to generate, utilize, display, or learn complex topographical maps or other advanced user interface mechanisms or vector-based algorithms.

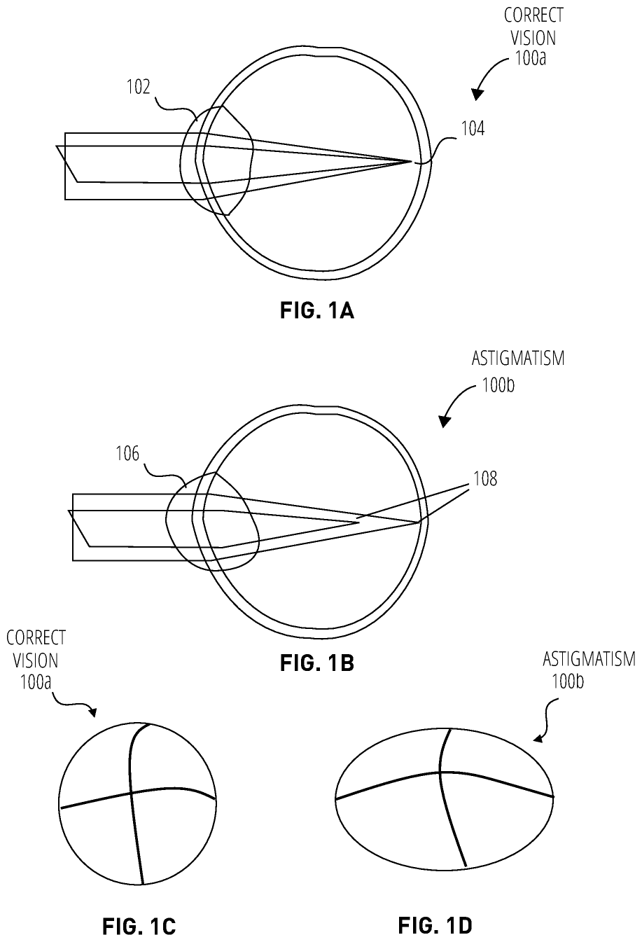

Astigmatism is most often caused by an ellipsoid (football-shaped) cornea or lens rather than a normal, spherically shaped cornea or lens. Less often, it is due to an irregular-shaped or displaced crystalline lens or corneal surface abnormality, such as a corneal scar. As depicted in FIG. 1A and FIG. 1C, substantially correct vision 100a is achieved by a spherical cornea 102 with a single focal point 104. As depicted in FIG. 1B and FIG. 1D, astigmatism 100b results from an oval cornea 106 that causes a split focal point 108.

With astigmatism 100b, light enters the eye, refracts, and comes to multiple points of focus, each taking place at different locations in the eye. The multiple focal points cause blurred vision.

Regular astigmatism 100b is the most common form of astigmatism resulting from the cornea having an ellipsoid shape rather than a spherical shape. The radius of curvature of an ellipsoid cornea varies along the meridians of the cornea.

The principal meridians (true vertical and true horizontal) of an oval cornea are substantially perpendicular and one meridian has a steeper gradient than the other. To correct for the resulting astigmatism, using spherocylinder lenses (lenses that include a spherical power, cylinder power, and an axis), rigid spherical contact lenses, toric rigid contacts, toric soft contact lenses, and LASIK or other refractive surgeries may be utilized. Intraocular lenses (IOLs) may also be implanted to correct astigmatism.

Spherical lenses have a single dioptric power, invariant radius of curvature, and a single point of focus. They exhibit equal power in all meridians of the lens. Spherical lenses correct vision for myopia and hyperopia but do not correct vision for astigmatism.

Cylindrical lens surfaces exhibit maximum power along one axis and no power along the axis orthogonal to maximum power axis. Astigmatism is corrected by lenses that have a cylinder component.

Spherocylinder lenses exhibit a spherical power and a cylinder power. The front surface of the lens is spherical and the back surface is cylindrical. The sphere power exhibits along one axis and the sphere and cylinder power combined exhibit orthogonally to this axis. Spherocylinder lenses are toric lenses with varying powers along all of the meridians.

Metrics for astigmatism correction include spherical power, cylinder power, and axis. The axis designates the meridian of the lens that only has the sphere power in effect with a number from 1 to 180; the full cylinder power is located 90 degrees away from the axis.

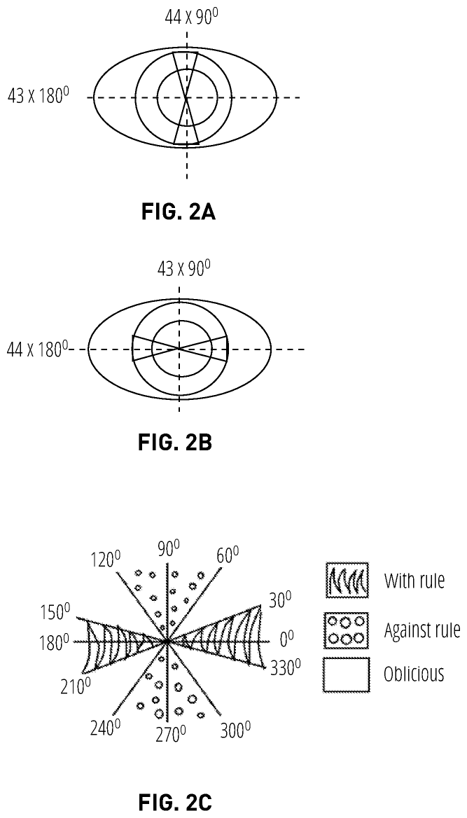

Referencing FIG. 2A, FIG. 2B, and FIG. 2C, astigmatism may be determined according to meridians of the cornea. One meridian comprises a line connected vertically from the 12 o'clock to six o'clock position: this is the vertical meridian and approximately the 90-degree axis. A line from three to nine o'clock is the horizontal meridian and approximately the 180-degree axis. With astigmatism, the steepest and flattest meridians of the eye are called the principal meridians. The amount of astigmatism is equal to the difference in refracting power of the two principal meridians.

“With-the-rule” astigmatism occurs when the vertical meridian of the cornea is steepest. Consider a football shape lying on its side, and the vertical meridian of the football is the steepest curve.

For these cases, lenses may be fabricated with a minus cylinder placed in the horizontal axis. Placing a minus cylinder in the horizontal axis allows the horizontal meridian to become steeper, thereby neutralizing or balancing the steepness of the vertical meridian. Lenses to correct this type of astigmatism may comprise an axis within 30 degrees of 180, so the axis falls between 001 to 030 or from 150 to 180.

“Against-the-rule” astigmatism occurs when the horizontal meridian of the cornea is steepest—the horizontal meridian of the football is the steepest curve. For these cases, the minus cylinder is placed in the vertical axis; the vertical meridian then becomes steeper and thus neutralizes or balances the steepness of the horizontal meridian. For these cases, lenses may be fabricated with an axis within 30 degrees of 090, so the axis falls between 060 to 120 or 240 to 300.

Oblique astigmatism occurs when the steepest curve of the cornea isn't in the vertical or horizontal meridians. It is rather in an oblique meridian between 120 and 150 degrees and 30 and 60 degrees. Lenses to correct for oblique astigmatism may comprise an axis that is not within 30 degrees of 090 and not within 30 degrees of 180.

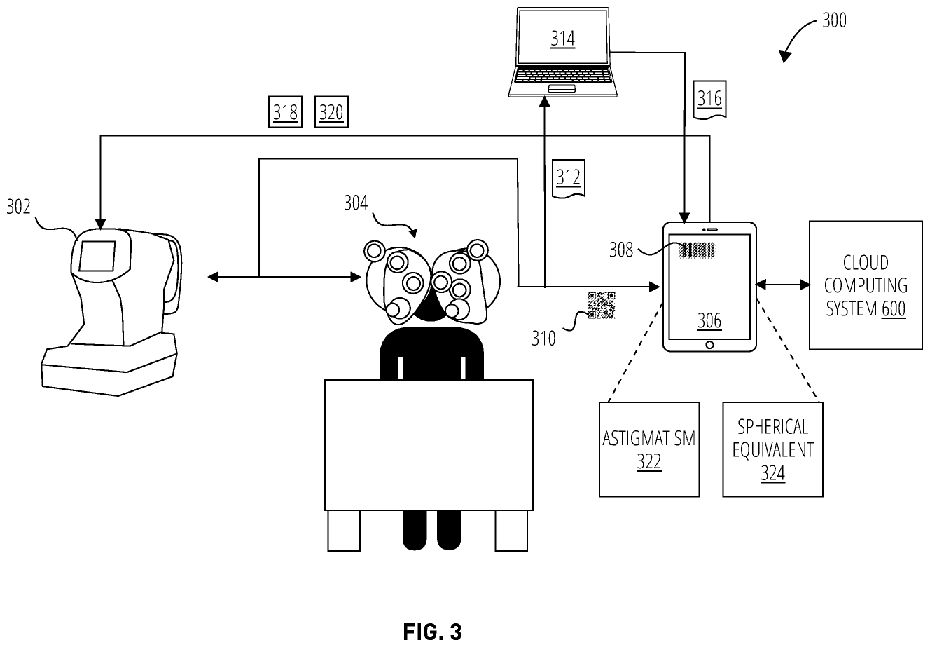

FIG. 3 depicts a vision analysis system 300 in one embodiment. The vision analysis system 300 comprises a an autorefractor 302, a phoroptor 304, and a computing device 306. The autorefractor 302 is a computer-controlled machine used during an eye examination to provide an objective measurement of a person's refractive error and prescription for lenses. This is achieved by measuring how light is changed as it enters a person's eye.

The autorefractor 302 may typically calculate the vision correction a patient needs (refraction) by using sensors that detect the reflections from a cone of infrared light. These reflections are used to determine the size and shape of a ring in the retina which is located in the posterior part of the eye. By measuring this zone, the autorefractor can determine when a patient's eye properly focuses an image. The instrument changes its magnification until the image comes into focus. The process is repeated in at least three meridians of the eye and the autorefractor 302 calculates the refraction of the eye, sphere, cylinder and axis.

This process is often used to provide the starting point for the vision professional in subjective refraction tests, in which lenses are switched in and out of the phoroptor 304 and the patient is asked “which looks better” while looking at an eye chart. This feedback refines the metrics for the lens prescription to more optimum values for the patient.

The phoroptor 304, also called a “refractor”, comprises different lenses used for refraction of the eye during sight testing, to measure an individual's refractive error. It may also be used to measure the patients' phorias and ductions, which are characteristics of binocularity. The phoroptor 304 may be operated manually, or may be automated.

Typically, the patient sits behind the phoroptor 304, and looks through it at an eye chart placed at optical infinity (20 feet or 6 metres), then at near (16 inches or 40 centimetres) for individuals needing reading glasses. The eye care professional then changes lenses and other settings, while asking the patient for subjective feedback on which settings gave the best vision. The patient's habitual prescription or the autorefractor 302 may be used to provide initial settings for the phoroptor 304.

The autorefractor 302 and/or phoroptor 304 may communicate a patient id (e.g., as a barcode 308) and measurement results (e.g., as a QR code 310) to an app on the computing device 306 (e.g., a cell phone). The autorefractor 302/phoroptor 304 may also communicate measurement results (e.g., as an XML file 312) to a data storage device 314 such as a laptop computer and/or cloud computing system 600, and the computing device 306 may access the stored XML file 316 for measurements corresponding to the patient identified by the barcode 308 or other patient id. The app on the computing device 306, and/or the data storage device 314, may communicate astigmatism metric algorithm results 318 and spherical equivalent metric algorithm results 320 for a patient, or group of patients having some common characteristic(s), to the cloud computing system 600 and/or back to the autorefractor 302/phoroptor 304.

The refraction derived from the autorefractor 302 and phoroptor 304 comprises three components:
Sphere
Cylinder
Axis

The spherical equivalent may be calculated by adding half the cylinder (cyl) to the sphere (sph): SEQ=sph+½ cyl.

Two types of cylinder may be applied for correcting astigmatism, referred to herein as “positive cylinder” and “negative cylinder”. Both may be used for correcting astigmatism, where positive cylinder uses positive diopters and the negative cylinder uses negative diopters. A diopter is a unit of refractive power that is equal to the reciprocal of the focal length (in meters) of a given lens.

The definition of “against the rule” and “with the rule” may vary depending in the cylinder used. With positive cylinder the more highly curved axis defines the rule. With negative cylinder the flatter axis defines the rule. Although they define the rule along different axes, both approaches produce similar results for characterizing the astigmatism. The type of cylinder utilized may be configurable by a user of the app or application of the computing device 306.

Additional independent variables may be associated with the metrics for astigmatism and spherical equivalent. These variables may be utilized to filter results and/or direct quality control feedback along particular physical vectors. For example:

TABLE 1

| | |
|---|---|
| Intraocular lens | Brand/Model |
| | Toric or non toric |
| | Multifocal or monofocal |
| Patient | Age |
| | Gender |
| | Comorbidities |
| | Eye (left, right) |
| Effective lens position | |
| IOL Tilt | |
| Biometer | Anterion |
| | IOL master |
| | Ultrasound |
| Special Equipment Used | Femtosecond Laser |
| | Optiwave Refractive Analysis |
| Surgeon | |
| Complication | Intraoperative |
| | Postoperative |

The computing device 306 may execute an astigmatism metric algorithm 322 and/or spherical equivalent metric algorithm 324 that each generate a small (<10) set of discrete metric values each corresponding to ranges of residual refractive error. These metrics may be applied back to machine settings for the different independent variables (Table 1) to improve future lens designs and thus patient outcomes.

The astigmatism metric algorithm 322 and spherical equivalent metric algorithm 324 may in one embodiment generate metric values from the set {1, 2, 3, 4, 5} determined by an amount of residual refractive error post-refractive surgery. The refractive surgery may be corneal and intraocular (e.g. Phacoemulsification, LASIK, PRK, ICL).

For residual cylinder computation from astigmatism, the metric in one embodiment is determined according to:

Algorithm 1

| | Cylinder | | | |
|---|---|---|---|---|
| | against the rule | | Cylinder With | |
| Metric | and obliques | | the rule | |
| Value | From | To | From | To |
| 5 | 0 | −0.25 | 0 | −0.5 |
| 4 | −0.26 | −0.5 | −0.51 | −1 |
| 3 | −0.51 | −0.75 | −1.01 | −1.25 |
| 2 | −0.76 | −1 | −1.26 | −1.5 |
| 1 | >−1.01 | | >−1.51 | |

In another embodiment:

Algorithm 2

| | Cylinder | | | |
|---|---|---|---|---|
| | against the rule | | Cylinder With | |
| Metric | and obliques | | the rule | |
| Value | From | To | From | To |
| 5 | 0 | 0.25 | 0 | 0.5 |
| 4 | 0.26 | 0.5 | 0.51 | 1 |
| 3 | 0.51 | 1 | 1.1 | 1.5 |
| 2 | 1.1 | | 1.51 | 2 |
| 1 | >2 | | >2 | |

Although depicted as either positive or negative values, the same tiers apply when the metrics are all made positive (or negative). Generally, Algorithms 1 or 2 may be carried out for more or fewer discrete ranges (tiers) of the metric. The tiers may correlate to levels of human visual distance acuity (e.g., 20/20, 20/25, 20/30, 20/40, etc.) The upper and/or lower range values of any one or more of the metrics may, according to the embodiment, vary by up to ±15%.

“With the rule” herein refers to the axis of the positive cylinder in a pair of glasses being oriented at 90 degrees)(+−30°; every other axis is oblique or, if oriented at 180 degrees, is against the rule (+−30).

For Spherical Equivalent (SEQ) from residual astigmatism, the metric in one embodiment is determined according to:

Algorithm 3

| Metric Value | SEQ |
|---|---|
| 5 | 0 to +−.0.25 |
| 4 | +−.0.25 to +−0.50 |
| 3 | +−0.50 to +−0.75 |
| 2 | +−0.75 to +−1.00 |
| 1 | >+−1.01 |

Generally, Algorithm 3 may be carried out for more or fewer discrete ranges (tiers) of the metric. The upper and/or lower range values of any one or more of the metrics may, according to the embodiment, vary by up to ±15%.

By way of these algorithms, a particular visual acuity level/residual cylinder (astigmatism) may be equated/correlated to a particular spherical equivalent level.

In one embodiment, an app or application (which may be local to the user's computer, or cloud-based) executes embodiments of the algorithms above, based on post-surgical inputs comprising residual manifest refraction, the intraocular lens used (if applicable), the formula used for calculating the intraocular lens (if applicable), and potentially other variables (see below). The evaluation by the algorithms may be performed at least six weeks post-surgery.

Metrics may be generated for individual patients, for classes of patients (patients having one or more common characteristics), or for all patients. The metrics may be further refined for patients of a specific surgeon, or group of surgeons, or for a surgical center, or for a group of surgical centers.

Metrics may be organized and/or filtered according to the intraocular lens used in a surgery, the formula used for calculating the intraocular lens, the use of particular equipment in the surgery (e.g. femtosecond laser), and/or for surgeries performed in a period of time. Metrics may be evaluated to rank the performance of different practitioners, lenses, and process variables.

Table 2 below depicts an example application of the algorithms described above to produce ranking metrics.

TABLE 2

| | | | | | | Rank | Rank | Rank |
|---|---|---|---|---|---|---|---|---|
| Eye | Sphere | Cyl | Axis | Rule | SEQ | Cyl | SEQ | Total |
| Right | 0 | −0.25 | 149 | Against | −0.125 | 5 | 5 | 5 |
| | | | | the Rule | | | | |
| Left | −0.5 | −0.25 | 86 | Against | −0.625 | 5 | 3 | 4 |
| | | | | the Rule | | | | |
| Right | 0 | −0.5 | 123 | Against | −0.25 | 4 | 5 | 4.5 |
| | | | | the Rule | | | | |
| Left | 0 | −0.25 | 111 | Against | −0.125 | 5 | 5 | 5 |
| | | | | the Rule | | | | |
| Left | 0.25 | −0.25 | 104 | Against | 0.125 | 5 | 5 | 5 |
| | | | | the Rule | | | | |
| Right | 0.25 | −0.5 | 114 | Against | 0 | 4 | 5 | 4.5 |
| | | | | the Rule | | | | |

The Ranking Cylinder is the ranking result from the algorithm depending of the residual astigmatism. For example, a 0.5 ‘With the Rule’ measurement corresponds to a Ranking Cylinder value of 5. A 0.5 ‘Against the Rule’ measure corresponds to a Ranking Cylinder value of 4. The value of the Ranking SEQ is determined in similar fashion from the Spherical Equivalent ranking algorithm.

Table 3 below depicts additional tags that may be applied to the rankings for categorization and control purposes:

TABLE 3

| | Toric | | | Refractive | | Measure | |
|---|---|---|---|---|---|---|---|
| Lens | Calc | Formula | Biometer | Surgery | UCDVA | Date | |
| PanOptix | Oculix | Barrett | Anterion | no | 20/25 | 6 | weeks |
| Toric | | Universal II | | | | | |
| PanOptix | Oculix | Barrett | Anterion | no | 20/25 | 6 | weeks |
| Toric | | Universal II | | | | | |
| PanOptix | Oculix | Kane | Anterion | no | 20/25 | 6 | months |
| PanOptix | Oculix | Barrett | Anterion | no | 20/20 | 6 | months |
| | | Universal II | | | | | |
| PanOptix | Oculix | Barrett | Anterion | no | 20/20 | 3 | months |
| Toric | | Universal II | | | | | |
| PanOptix | Oculix | Barrett | Anterion | no | 20/20 | 3 | months |
| | | Universal II | | | | | |

Here UCDVA refers to Uncorrected Distance Visual Acuity. Algorithms 1-3 result from and provide a correlation between residual astigmatism and the visual acuity. These algorithms provide a metric how much and what type of residual astigmatism is necessary for visual acuity to change.

Ratings (for residual astigmatism and SEQ) may be generated per patient individually (astigmatism and SEQ), globally (e.g., mean/average) for all patients or groups of patients sharing certain characteristics (age, gender, comorbidities, lens type etc.), and/or for a particular surgeon or center (e.g., mean/average).

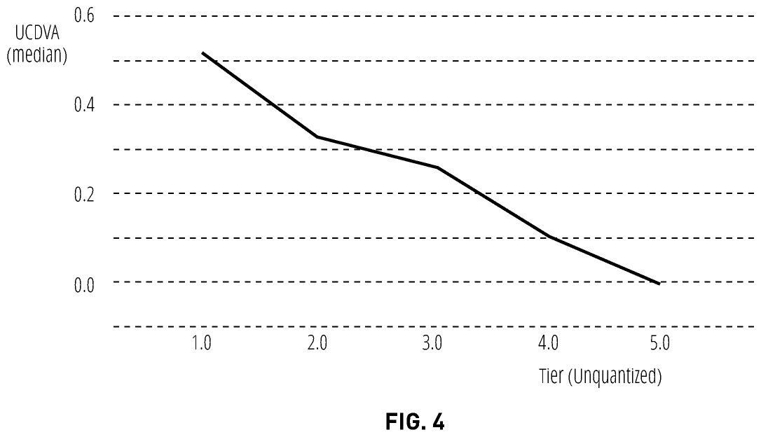

FIG. 4 depicts mapping of uncorrected distance visual acuity (UCDVA) to a (unquantized) metric control, in accordance with one embodiment. The mapping comprises a linear regression of median UCDVA, with R-squared=0.74 and a Spearman Correlation Coefficient of −0.774. In conjunction with Algorithms 1-3, correlation between quantized metric controls, visual acuity, residual cylinder, and residual spherical equivalent may thereby be established.

Due to their reduced complexity, the disclosed mechanisms may be operationally more robust than conventional approaches to lens design, selection, and evaluation and may exhibit improved performance and/or reliability, and may reduce the likelihood of mistakes. The disclosed mechanisms also increase the likelihood that practitioners will reliably perform post-operative evaluation. For these same reasons the mechanisms may also improve the consistency of lens design, selection, and evaluation methodologies across a variety of eye surgery practices.

The algorithms disclosed herein, or particular components thereof, may in some embodiments be implemented as software comprising instructions executed on one or more programmable device. By way of example, components of the disclosed systems (algorithms, user interfaces) may be implemented as an application, an app, drivers, or services. In one particular embodiment, aspects of the system are implemented as service(s) that execute as one or more processes, modules, subroutines, or tasks on a server system so as to provide the described capabilities to one or more client devices over a network. However the system need not necessarily be accessed over a network and could, in some embodiments, be implemented by one or more app or applications on a single device or distributed between a mobile device and a computer, for example.

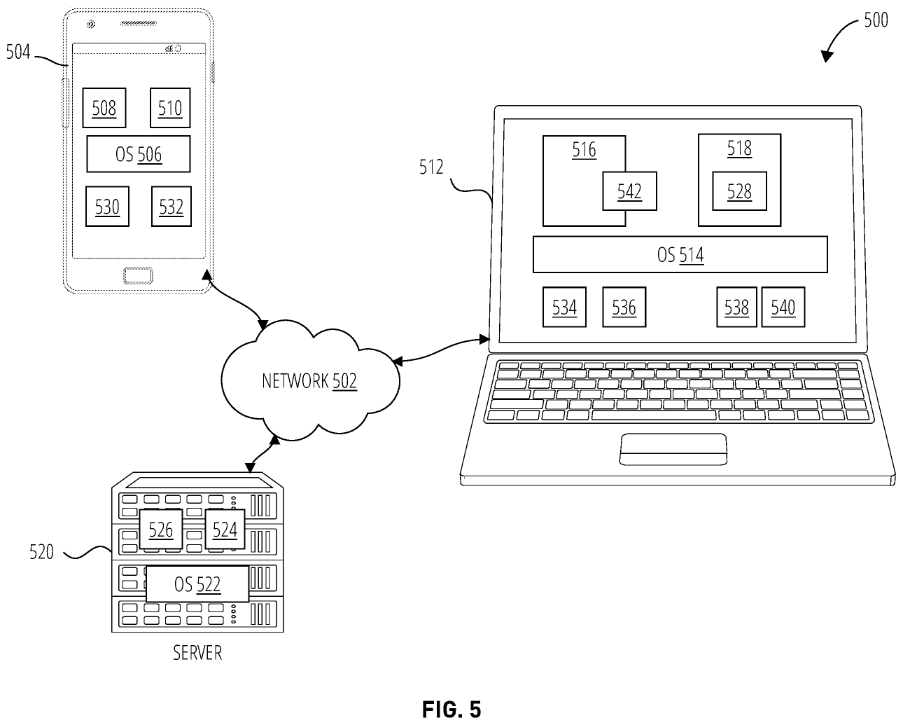

Referring to FIG. 5, a client server network configuration 500 in which the disclosed mechanisms may operate includes various computer hardware devices and software modules coupled by a network 502 in one embodiment. For example one or more of the algorithms may execute in a cloud computing system and a user interface to the cloud computing system may execute on a mobile device. In another example, one or more of the algorithms and user interface may execute locally on the laptop or mobile devices or desktop systems of multiple practitioners, and a cloud computing system may collect and analyze (rank, filter etc.) metrics received from the practitioners' devices. Each device includes a native operating system, typically pre-installed on its non-volatile RAM, and a variety of software applications or apps for performing various functions.

The mobile programmable device 504 comprises a native operating system 506 and various apps (e.g., app 508 and app 510). A computer 512 also includes an operating system 514 that may include one or more library of native routines to run executable software on that device. The computer 512 also includes various executable applications (e.g., application 516 and application 518). The mobile programmable device 504 and computer 512 are configured as clients on the network 502. A server 520 is also provided and includes an operating system 522 with native routines specific to providing a service (e.g., service 524 and service 526) available to the networked clients in this configuration.

As is well known in the art, an application, an app, or a service may be created by first writing computer code to form a computer program, which typically comprises one or more computer code sections or modules. Computer code may comprise instructions in many forms, including source code, assembly code, object code, executable code, and machine language. Computer programs often implement mathematical functions or algorithms and may implement or utilize one or more application program interfaces.

A compiler is typically used to transform source code into object code and thereafter a linker combines object code files into an executable application, recognized by those skilled in the art as an “executable”. The distinct file comprising the executable would then be available for use by the computer 512, mobile programmable device 504, and/or server 520. Any of these devices may employ a loader to place the executable and any associated library in memory for execution. The operating system executes the program by passing control to the loaded program code, creating a task or process. An alternate means of executing an application or app involves the use of an interpreter (e.g., interpreter 528).

In addition to executing applications (“apps”) and services, the operating system is also typically employed to execute drivers to perform common tasks such as connecting to third-party hardware devices (e.g., printers, displays, input devices), storing data, interpreting commands, and extending the capabilities of applications. For example, a driver 530 or driver 532 on the mobile programmable device 504 or computer 512 (e.g., driver 534 and driver 536) might enable wireless headphones to be used for audio output(s) and a camera to be used for video inputs. Any of the devices may read and write data from and to files (e.g,. file 538 or file 540) and applications or apps may utilize one or more plug-in (e.g., plug-in 542) to extend their capabilities (e.g., to encode or decode video files).

The network 502 in the client server network configuration 500 can be of a type understood by those skilled in the art, including a Local Area Network (LAN), Wide Area Network (WAN), Transmission Communication Protocol/Internet Protocol (TCP/IP) network, and so forth. These protocols used by the network 502 dictate the mechanisms by which data is exchanged between devices.

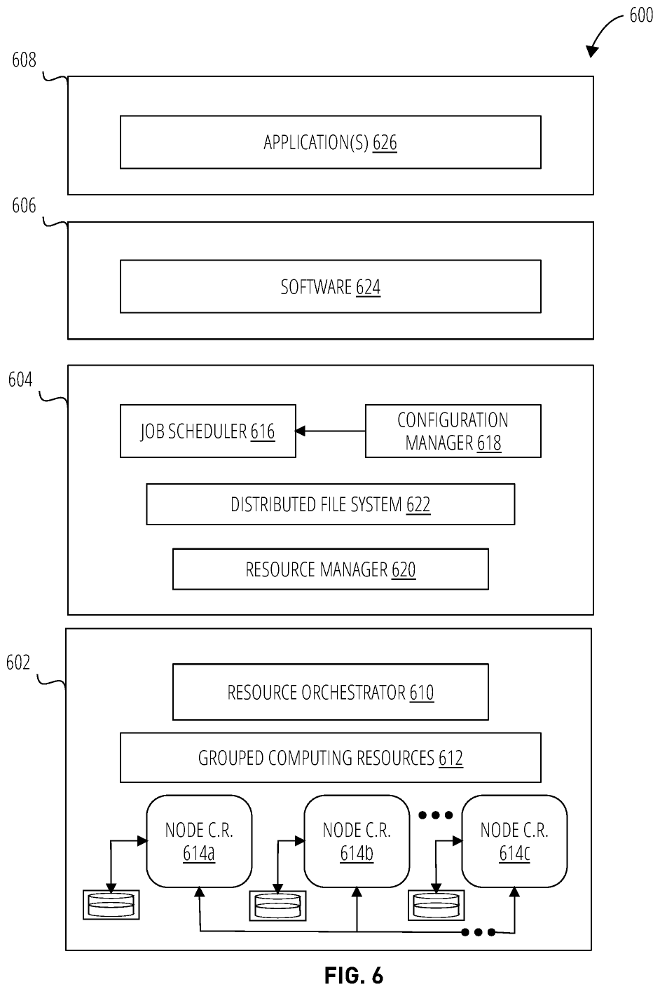

FIG. 6 depicts an exemplary cloud computing system 600, in accordance with at least one embodiment. In at least one embodiment, cloud computing system 600 includes, without limitation, a data center infrastructure layer 602, a framework layer 604, software layer 606, and an application layer 608.

Logic of the cloud computing system 600 may operate cooperatively with an app or application of a mobile programmable device 504 or other practitioner device (e.g., data storage device 314) to provide one or more of: configuring a rule (e.g., “with the rule” or “against the rule”; configuring a cylinder comprising one of a “positive cylinder” and a “negative cylinder”; generating a ruled cylinder by apply the rule to the cylinder; utilizing the ruled cylinder in one or both of a astigmatism metric algorithm and spherical equivalent metric algorithm to generate a discrete metric values each corresponding to ranges of residual refractive error; and configuring lens settings based on the discrete metric values for one or more independent variables to improve future lens designs and thus patient surgical outcomes; and applying the lens settings to selection or manufacture of a lens.

As noted previous, the metric values may in one embodiment be drawn from the set {1, 2, 3, 4, 5} wherein refractive error is derived from a corneal or intraocular surgery. Each discrete metric value to a level of human visual distance acuity.

The cloud computing system 600 may provide one or more of filtering and ranking the metrics from a single practitioner, a group of practitioners, one or more patient characteristics, the type of intraocular lens used in a surgery, the formula used for calculating the intraocular lens characteristics, and practitioner process variables (e.g., surgical procedural characteristics).

The cloud computing system 600 may comprise logic to generate ratings (for residual astigmatism and SEQ) may be generated per patient individually (astigmatism and SEQ), globally (e.g., mean/average) for all patients or groups of patients sharing certain characteristics (age, gender, comorbidities, lens type etc.), and/or for a particular surgeon or center (e.g., mean/average).

In at least one embodiment, as depicted in FIG. 6, data center infrastructure layer 602 may include a resource orchestrator 610, grouped computing resources 612, and node computing resources (“node C. R. s”) Node C. R. 614a, Node C. R. 614b, Node C. R. 614c, . . . node C. R. N), where “N” represents any whole, positive integer. In at least one embodiment, node C. R. s may include, but are not limited to, any number of central processing units (“CPUs”) or other processors (including accelerators, field programmable gate arrays (“FPGAs”), graphics processors, etc.), memory devices (e.g, dynamic read-only memory), storage devices (e.g., solid state or disk drives), network input/output (“NW I/O”) devices, network switches, virtual machines (“VMs”), power modules, and cooling modules, etc. In at least one embodiment, one or more node C. R. s from among node C. R. s may be a server having one or more of above-mentioned computing resources.

In at least one embodiment, grouped computing resources 612 may include separate groupings of node C. R. s housed within one or more racks (not shown), or many racks housed in data centers at various geographical locations (also not shown). Separate groupings of node C. R. s within grouped computing resources 612 may include grouped compute, network, memory or storage resources that may be configured or allocated to support one or more workloads. In at least one embodiment, several node C. R. s including CPUs or processors may grouped within one or more racks to provide compute resources to support one or more workloads. In at least one embodiment, one or more racks may also include any number of power modules, cooling modules, and network switches, in any combination.

In at least one embodiment, resource orchestrator 610 may configure or otherwise control one or more node C. R. s and/or grouped computing resources 612. In at least one embodiment, resource orchestrator 610 may include a software design infrastructure (“SDI”) management entity for cloud computing system 600. In at least one embodiment, resource orchestrator 610 may include hardware, software or some combination thereof.

In at least one embodiment, as depicted in FIG. 6, framework layer 604 includes, without limitation, a job scheduler 616, a configuration manager 618, a resource manager 620, and a distributed file system 622. In at least one embodiment, framework layer 604 may include a framework to support software 624 of software layer 606 and/or one or more application(s) 626 of application layer 220. In at least one embodiment, software 624 or application(s) 626 may respectively include web-based service software or applications, such as those provided by Amazon Web Services, Google Cloud and Microsoft Azure. In at least one embodiment, framework layer 604 may be, but is not limited to, a type of free and open-source software web application framework such as Apache Spark™ (hereinafter “Spark”) that may utilize a distributed file system 622 for large-scale data processing (e.g., “big data”). In at least one embodiment, job scheduler 616 may include a Spark driver to facilitate scheduling of workloads supported by various layers of cloud computing system 600. In at least one embodiment, configuration manager 618 may be capable of configuring different layers such as software layer 606 and framework layer 604, including Spark and distributed file system 622 for supporting large-scale data processing. In at least one embodiment, resource manager 620 may be capable of managing clustered or grouped computing resources mapped to or allocated for support of distributed file system 622 and distributed file system 622. In at least one embodiment, clustered or grouped computing resources may include grouped computing resources 612 at data center infrastructure layer 602. In at least one embodiment, resource manager 620 may coordinate with resource orchestrator 610 to manage these mapped or allocated computing resources.

In at least one embodiment, software 624 included in software layer 606 may include software used by at least portions of node C. R. s, grouped computing resources 612, and/or distributed file system 622 of framework layer 604. One or more types of software may include, but are not limited to, Internet web page search software, e-mail virus scan software, database software, and streaming video content software.

In at least one embodiment, application(s) 626 included in application layer 608 may include one or more types of applications used by at least portions of node C. R. s, grouped computing resources 612, and/or distributed file system 622 of framework layer 604. In at least one or more types of applications may include, without limitation, CUDA applications, 5G network applications, artificial intelligence application, data center applications, and/or variations thereof.

In at least one embodiment, any of configuration manager 618, resource manager 620, and resource orchestrator 610 may implement any number and type of self-modifying actions based on any amount and type of data acquired in any technically feasible fashion. In at least one embodiment, self-modifying actions may relieve a data center operator of cloud computing system 600 from making possibly bad configuration decisions and possibly avoiding underutilized and/or poor performing portions of a data center.

### Machine Embodiments

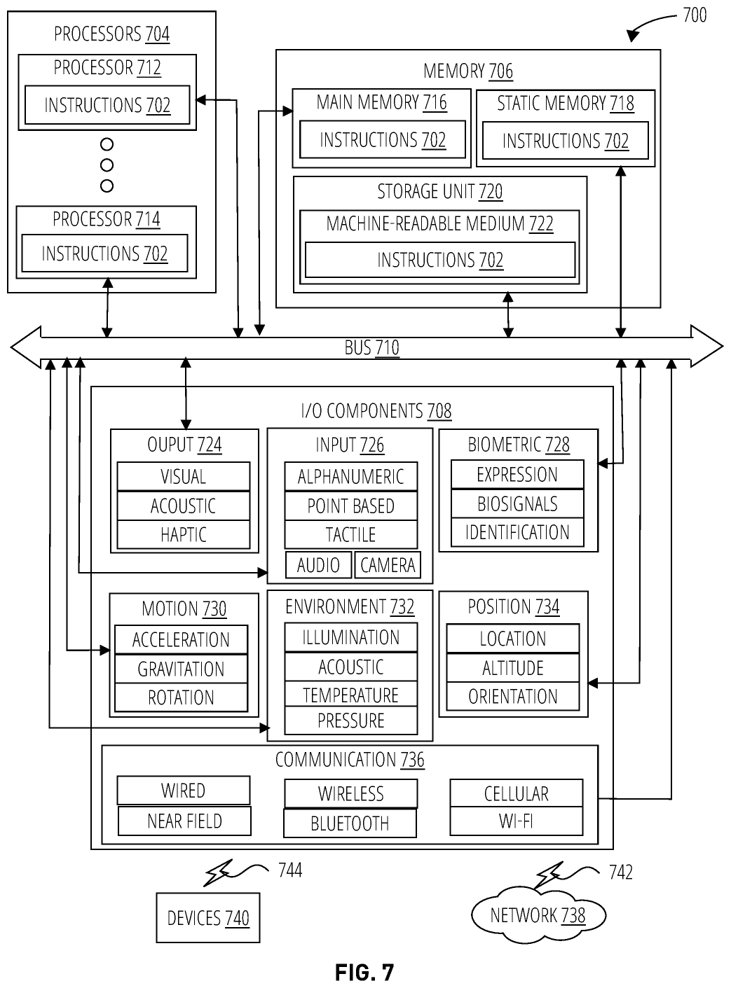

FIG. 7 depicts a diagrammatic representation of a machine 700 in the form of a computer system within which logic may be implemented to cause the machine to perform any one or more of the functions or methods disclosed herein, according to an example embodiment.

Specifically, FIG. 7 depicts a machine 700 comprising instructions 702 (e.g., a program, an application, an applet, an app, or other executable code) for causing the machine 700 to perform any one or more of the functions or methods discussed herein. For example the instructions 702 may cause the machine 700 to carry out embodiments of the astigmatism and spherical equivalent algorithms disclosed herein. The instructions 702 configure a general, non-programmed machine into a particular machine 700 programmed to carry out said functions and/or methods.

In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 702, sequentially or otherwise, that specify actions to be taken by the machine 700. Further, while only a single machine 700 is depicted, the term “machine” shall also be taken to include a collection of machines that individually or jointly execute the instructions 702 to perform any one or more of the methodologies or subsets thereof discussed herein.

The machine 700 may include processors 704, memory 706, and I/O components 708, which may be configured to communicate with each other such as via one or more bus 710. In an example embodiment, the processors 704 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, one or more processor (e.g., processor 712 and processor 714) to execute the instructions 702. The term “processor” is intended to include multi-core processors that may comprise two or more independent processors (sometimes referred to as “cores”) that may execute instructions contemporaneously. Although FIG. 7 depicts multiple processors 704, the machine 700 may include a single processor with a single core, a single processor with multiple cores (e.g., a multi-core processor), multiple processors with a single core, multiple processors with multiples cores, or any combination thereof.

The memory 706 may include one or more of a main memory 716, a static memory 718, and a storage unit 720, each accessible to the processors 704 such as via the bus 710. The main memory 716, the static memory 718, and storage unit 720 may be utilized, individually or in combination, to store the instructions 702 embodying any one or more of the functionality described herein. The instructions 702 may reside, completely or partially, within the main memory 716, within the static memory 718, within a machine-readable medium 722 within the storage unit 720, within at least one of the processors 704 (e.g., within the processor's cache memory), or any suitable combination thereof, during execution thereof by the machine 700.

The I/O components 708 may include a wide variety of components to receive input, provide output, produce output, transmit information, exchange information, capture measurements, and so on. The specific I/O components 708 that are included in a particular machine will depend on the type of machine. For example, portable machines such as mobile phones will likely include a touch input device or other such input mechanisms, while a headless server machine will likely not include such a touch input device. It will be appreciated that the I/O components 708 may include many other components that are not shown in FIG. 7. The I/O components 708 are grouped according to functionality merely for simplifying the following discussion and the grouping is in no way limiting. In various example embodiments, the I/O components 708 may include output components 724 and input components 726. The output components 724 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 726 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), one or more cameras for capturing still images and video, and the like.

In further example embodiments, the I/O components 708 may include biometric components 728, motion components 730, environmental components 732, or position components 734, among a wide array of possibilities. For example, the biometric components 728 may include components to detect expressions (e.g., hand expressions, facial expressions, vocal expressions, body gestures, or eye tracking), measure bio-signals (e.g., blood pressure, heart rate, body temperature, perspiration, or brain waves), identify a person (e.g., voice identification, retinal identification, facial identification, fingerprint identification, or electroencephalogram-based identification), and the like. The motion components 730 may include acceleration sensor components (e.g., accelerometer), gravitation sensor components, rotation sensor components (e.g., gyroscope), and so forth. The environmental components 732 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 734 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 708 may include communication components 736 operable to couple the machine 700 to a network 738 or devices 740 via a coupling 742 and a coupling 744, respectively. For example, the communication components 736 may include a network interface component or another suitable device to interface with the network 738. In further examples, the communication components 736 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 740 may be another machine or any of a wide variety of peripheral devices (e.g., a peripheral device coupled via a USB).

Moreover, the communication components 736 may detect identifiers or include components operable to detect identifiers. For example, the communication components 736 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 736, such as location via Internet Protocol (IP) geolocation, location via Wi-Fi® signal triangulation, location via detecting an NFC beacon signal that may indicate a particular location, and so forth.

### Instruction and Data Storage Medium Embodiments

The various memories (i.e., memory 706, main memory 716, static memory 718, and/or memory of the processors 704) and/or storage unit 720 may store one or more sets of instructions and data structures (e.g., software) embodying or utilized by any one or more of the methodologies or functions described herein. These instructions (e.g., the instructions 702), when executed by processors 704, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors and internal or external to computer systems. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such intangible media, at least some of which are covered under the term “signal medium” discussed below.

Some aspects of the described subject matter may in some embodiments be implemented as computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular data structures in memory. The subject matter of this application may be practiced in a variety of system configurations, including hand-held devices, consumer electronics, general-purpose computers, more specialty computing devices, etc. The subject matter may also be practiced in distributed computing environments where tasks are performed by remote-processing devices that are linked through a communications network.

### Communication Network Embodiments

In various example embodiments, one or more portions of the network 738 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 738 or a portion of the network 738 may include a wireless or cellular network, and the coupling 742 may be a Code Division Multiple Access (CDMA) connection, a Global System for Mobile communications (GSM) connection, or another type of cellular or wireless coupling. In this example, the coupling 742 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 702 and/or data generated by or received and processed by the instructions 702 may be transmitted or received over the network 738 using a transmission medium via a network interface device (e.g., a network interface component included in the communication components 736) and utilizing any one of a number of well-known transfer protocols (e.g., hypertext transfer protocol (HTTP)). Similarly, the instructions 702 may be transmitted or received using a transmission medium via the coupling 744 (e.g., a peer-to-peer coupling) to the devices 740. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 702 for execution by the machine 700, and/or data generated by execution of the instructions 702, and/or data to be operated on during execution of the instructions 702, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

### LISTING OF DRAWING ELEMENTS

100a correct vision
100b astigmatism
102 spherical cornea
104 single focal point
106 oval cornea
108 split focal point
300 vision analysis system
302 autorefractor
304 phoroptor
306 computing device
308 barcode
310 QR code
312 XML file
314 data storage device
316 XML file
318 astigmatism metric algorithm results
320 spherical equivalent metric algorithm results
322 astigmatism metric algorithm
324 spherical equivalent metric algorithm
500 client server network configuration
502 network
504 mobile programmable device
506 operating system
508 app
510 app
512 computer
514 operating system
516 application
518 application
520 server
522 operating system
524 service
526 service
528 interpreter
530 driver
532 driver
534 driver
536 driver
538 file
540 file
542 plug-in
600 cloud computing system
602 data center infrastructure layer
604 framework layer
606 software layer
608 application layer
610 resource orchestrator
612 grouped computing resources
614a node C. R.
614b node C. R.
614c node C. R.
616 job scheduler
618 configuration manager
620 resource manager
622 distributed file system
624 software
626 application(s)
700 machine
702 instructions
704 processors
706 memory
708 I/O components
710 bus
712 processor
714 processor
716 main memory
718 static memory
720 storage unit
722 machine-readable medium
724 output components
726 input components
728 biometric components
730 motion components
732 environmental components
734 position components
736 communication components
738 network
740 devices
742 coupling
744 coupling

“Algorithm” refers to any set of instructions configured to cause a machine to carry out a particular function or process.

“App” refers to a type of application with limited functionality, most commonly associated with applications executed on mobile devices. Apps tend to have a more limited feature set and simpler user interface than applications as those terms are commonly understood in the art.

“Application” refers to any software that is executed on a device above a level of the operating system. An application will typically be loaded by the operating system for execution and will make function calls to the operating system for lower-level services. An application often has a user interface but this is not always the case. Therefore, the term ‘application’ includes background processes that execute at a higher level than the operating system.

“Application program interface” refers to instructions implementing entry points and return values to a module.

“Assembly code” refers to a low-level source code language comprising a strong correspondence between the source code statements and machine language instructions. Assembly code is converted into executable code by an assembler. The conversion process is referred to as assembly. Assembly language usually has one statement per machine language instruction, but comments and statements that are assembler directives, macros, and symbolic labels may also be supported.

“Compiled computer code” refers to object code or executable code derived by executing a source code compiler and/or subsequent tools such as a linker or loader.

“Compiler” refers to logic that transforms source code from a high-level programming language into object code or in some cases, into executable code.

“Computer code” refers to any of source code, object code, or executable code.

“Computer code section” refers to one or more instructions.

“Computer program” refers to another term for ‘application’ or ‘app’.

“Driver” refers to low-level logic, typically software, that controls components of a device. Drivers often control the interface between an operating system or application and input/output components or peripherals of a device, for example.

“Executable” refers to a file comprising executable code. If the executable code is not interpreted computer code, a loader is typically used to load the executable for execution by a programmable device.

“Executable code” refers to instructions in a ready-to-execute form by a programmable device. For example, source code instructions in non-interpreted execution environments are not executable code because they must usually first undergo compilation, linking, and loading by the operating system before they have the proper form for execution. Interpreted computer code may be considered executable code because it can be directly applied to a programmable device (an interpreter) for execution, even though the interpreter itself may further transform the interpreted computer code into machine language instructions.

“File” refers to a unitary package for storing, retrieving, and communicating data and/or instructions. A file is distinguished from other types of packaging by having associated management metadata utilized by the operating system to identify, characterize, and access the file.

“Instructions” refers to symbols representing commands for execution by a device using a processor, microprocessor, controller, interpreter, or other programmable logic. Broadly, ‘instructions’ can mean source code, object code, and executable code. ‘instructions’ herein is also meant to include commands embodied in programmable read-only memories (EPROM) or hard coded into hardware (e.g., ‘micro-code’) and like implementations wherein the instructions are configured into a machine memory or other hardware component at manufacturing time of a device.

“Interpreted computer code” refers to instructions in a form suitable for execution by an interpreter.

“Interpreter” refers to an interpreter is logic that directly executes instructions written in a source code scripting language, without requiring the instructions to a priori be compiled into machine language. An interpreter translates the instructions into another form, for example into machine language, or into calls to internal functions and/or calls to functions in other software modules.

“Library” refers to a collection of modules organized such that the functionality of all the modules may be included for use by software using references to the library in source code.

“Linker” refers to logic that inputs one or more object code files generated by a compiler or an assembler and combines them into a single executable, library, or other unified object code output. One implementation of a linker directs its output directly to machine memory as executable code (performing the function of a loader as well).

“Loader” refers to logic for loading programs and libraries. The loader is typically implemented by the operating system. A typical loader copies an executable into memory and prepares it for execution by performing certain transformations, such as on memory addresses.

“Machine language” refers to instructions in a form that is directly executable by a programmable device without further translation by a compiler, interpreter, or assembler. In digital devices, machine language instructions are typically sequences of ones and zeros.

“Module” refers to a computer code section having defined entry and exit points. Examples of modules are any software comprising an application program interface, drivers, libraries, functions, and subroutines.

“Object code” refers to the computer code output by a compiler or as an intermediate output of an interpreter. Object code often takes the form of machine language or an intermediate language such as register transfer language (RTL).

“Operating system” refers to logic, typically software, that supports a device's basic functions, such as scheduling tasks, managing files, executing applications, and interacting with peripheral devices. In normal parlance, an application is said to execute “above” the operating system, meaning that the operating system is necessary in order to load and execute the application and the application relies on modules of the operating system in most cases, not vice-versa. The operating system also typically intermediates between applications and drivers. Drivers are said to execute “below” the operating system because they intermediate between the operating system and hardware components or peripheral devices.

“Plug-in” refers to software that adds features to an existing computer program without rebuilding (e.g., changing or re-compiling) the computer program. Plug-ins are commonly used for example with Internet browser applications.

“Process” refers to software that is in the process of being executed on a device.

“Programmable device” refers to any logic (including hardware and software logic) who's operational behavior is configurable with instructions.

“Service” refers to a process configurable with one or more associated policies for use of the process. Services are commonly invoked on server devices by client devices, usually over a machine communication network such as the Internet. Many instances of a service may execute as different processes, each configured with a different or the same policies, each for a different client.

“Software” refers to logic implemented as instructions for controlling a programmable device or component of a device (e.g., a programmable processor, controller). Software can be source code, object code, executable code, machine language code. Unless otherwise indicated by context, software shall be understood to mean the embodiment of said code in a machine memory or hardware component, including “firmware” and micro-code.

“Source code” refers to a high-level textual computer language that requires either interpretation or compilation in order to be executed by a device.

“Subroutine” refers to a module configured to perform one or more calculations or other processes. In some contexts the term ‘subroutine’ refers to a module that does not return a value to the logic that invokes it, whereas a ‘function’ returns a value. However herein the term ‘subroutine’ is used synonymously with ‘function’.

“Task” refers to one or more operations that a process performs.

Various functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on. “Logic” refers to machine memory circuits and non-transitory machine readable media comprising machine-executable instructions (software and firmware), and/or circuitry (hardware) which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

Within this disclosure, different entities (which may variously be referred to as “units,” “circuits,” other components, etc.) may be described or claimed as “configured” to perform one or more tasks or operations. This formulation—[entity] configured to [perform one or more tasks]—is used herein to refer to structure (i.e., something physical, such as an electronic circuit). More specifically, this formulation is used to indicate that this structure is arranged to perform the one or more tasks during operation. A structure can be said to be “configured to” perform some task even if the structure is not currently being operated. A “credit distribution circuit configured to distribute credits to a plurality of processor cores” is intended to cover, for example, an integrated circuit that has circuitry that performs this function during operation, even if the integrated circuit in question is not currently being used (e.g., a power supply is not connected to it). Thus, an entity described or recited as “configured to” perform some task refers to something physical, such as a device, circuit, memory storing program instructions executable to implement the task, etc. This phrase is not used herein to refer to something intangible.

The term “configured to” is not intended to mean “configurable to.” An unprogrammed FPGA, for example, would not be considered to be “configured to” perform some specific function, although it may be “configurable to” perform that function after programming.

Reciting in the appended claims that a structure is “configured to” perform one or more tasks is expressly intended not to invoke 35 U.S.C. § 112(f) for that claim element. Accordingly, claims in this application that do not otherwise include the “means for” [performing a function] construct should not be interpreted under 35 U.S.C § 112(f).

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” describes one or more factors that trigger an effect. This phrase does not foreclose the possibility that additional factors may affect or otherwise trigger the effect. That is, an effect may be solely in response to those factors, or may be in response to the specified factors as well as other, unspecified factors. Consider the phrase “perform A in response to B.” This phrase specifies that B is a factor that triggers the performance of A. This phrase does not foreclose that performing A may also be in response to some other factor, such as C. This phrase is also intended to cover an embodiment in which A is performed solely in response to B.

As used herein, the terms “first,” “second,” etc. are used as labels for nouns that they precede, and do not imply any type of ordering (e.g., spatial, temporal, logical, etc.), unless stated otherwise. For example, in a register file having eight registers, the terms “first register” and “second register” can be used to refer to any two of the eight registers, and not, for example, just logical registers 0 and 1.

When used in the claims, the term “or” is used as an inclusive or and not as an exclusive or. For example, the phrase “at least one of x, y, or z” means any one of x, y, and z, as well as any combination thereof.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Having thus described illustrative embodiments in detail, it will be apparent that modifications and variations are possible without departing from the scope of the invention as claimed. The scope of inventive subject matter is not limited to the depicted embodiments but is rather set forth in the following Claims.

What is claimed is:

1. A method comprising:
configuring a rule comprising one of a “with the rule” and “against the rule”;
configuring a cylinder comprising one of a “positive cylinder” and a “negative cylinder”;
utilizing the rule and the cylinder in one or both of a residual astigmatism metric algorithm and spherical equivalent metric algorithm to generate a discrete metric values each corresponding to ranges of residual refractive error; and
configuring lens settings based on the discrete metric values.

2. The method of claim 1, further comprising:
associating the discrete metric values with one or more type of a type of lens or a patient characteristic.

3. The method of claim 1, wherein the metric values are from the set {1, 2, 3, 4, 5}.

4. The method of claim 1, wherein refractive error is derived for a corneal or intraocular surgery.

5. The method of claim 1, further comprising:
correlating each discrete metric value to a level of human visual distance acuity.

6. The method of claim 1, further comprising:
one or more of filtering and ranking the metrics according to the type of intraocular lens used in a surgery.

7. The method of claim 1, further comprising:
one or more of filtering and ranking the metrics according to the formula used for calculating the intraocular lens characteristics.

8. The method of claim 1, further comprising:
one or more of filtering and ranking the metrics according to practitioner process variables.

9. A system to correlate residual astigmatism or spherical equivalent with visual acuity for intraocular or corneal refractive surgery, the system comprising:
an autorefractor/phoropter to measure residual astigmatism in at least one eye of a patient;
a first quantization algorithm to translate the residual astigmatism into a first tier value for residual cylinder;
a second quantization algorithm to translate the residual astigmatism value into a second tier value for spherical equivalent;
logic to apply one or both of the first tier value and the second tier value to determine a visual acuity of the patient; and
applying the visual acuity to settings of a corrective lens for the patient.

10. A method for selecting a vision correcting lens, the method comprising:
computing a metric of astigmatism according to Algorithm 1 or Algorithm 2;
applying the metric as feedback along one or more of the physical vectors of Table 1; and
selecting the lens based on the feedback along the physical vector.

11. A method for selecting a vision correcting lens, the method comprising:
computing a spherical equivalent metric according to Algorithm 3;
apply the metric as feedback along one or more of the physical vectors of Table 1; and
selecting the lens based on the feedback along the physical vector.
